(19) United States
(12) Patent Application Publication
(10) Pub. No.: US 2006/0228304 A1
(43) Pub. Date: Oct. 12, 2006

(54) Liquid embolisate

(76) Inventor: Friedhelm Brassel, Bochum (DE)

Correspondence Address: KENYON & KENYON LLP, ONE BROADWAY, NEW YORK, NY, 10004, US

(21) Appl. No.: 10/541,277

(22) PCT Filed: Dec. 29, 2003
(86) PCT No.: PCT/EP03/14940

§ 371 Date: Jun. 8, 2006

(30) Foreign Application Priority Data
Dec. 30, 2002 (DE) 102 61 694.9

(51) Int. Cl. A61K 49/04; A61M 11/00

(52) U.S. Cl. 424/9.41; 128/200.23

(57) ABSTRACT

The invention relates to a liquid embolizate, especially for the occlusion of vascular malformations. Said liquid embolizate is made of: a) 20-80% v/v of an occlusion mixture which contains a zein emulsion in aqueous ethanol, b) 10-40% v/v of a radiopaque contrast medium in liquid form and c) 10-40% v/v of ethanol. The components (a), (b) and (c) are separated and are mixed immediately before application in order to form a homogeneous emulsion. The invention also relates to a method for the production of said embolizate, the use thereof, in addition to a medical kit containing said embolizate.

The invention relates to a liquid embolizate intended for the occlusion of vascular malformations with said embolizate in ready-to-apply condition forming a stable emulsion of high radiopaqueness and minor separation tendency.

The occlusion of vascular malformations with the aid of endovascular techniques has gained paramount importance in recent decades, particularly for the occlusion of arteriovenous fistulas and aneurisms even in intracranial areas. The arteriovenous short circuits encountered in this context are normally just congenital regional extensions within the capillary vessel sections between the arterial and venous systems which may arise in the form of a simple fistula or as vascular networks. Frequently, such arteriovenous short circuits develop in highly vascular tumors.

Aneurysms are protuberances in blood vessels which may develop as a result of a tissue weakness and over time show a tendency towards dilatation due to pressure exerted by the flow of blood. As the vessel wall becomes increasingly thinner there is a risk of tearing associated with serious complications which when occurring intracranially may frequently lead to the death or severe disablement of patients.

In recent years numerous methods have been developed with a view to sclerotizing arteriovenous malformations of this type based on the endovascular injection of embolization substances. Basically, two different groups of embolization materials are employed.

On the one hand, this includes so-called liquid embolizates (strong alcohol, acrylates, fibrin glue, Aethoxysklerol®, Ethibloc®) which when injected into the vessel material causes the embolization material to set quickly when in contact with blood or an excessive irritation of the vessel wall resulting in the vessel volume to become obliterated.

On the other hand, particulate embolizates are introduced, for example in the form of small particles such as polyvinyl alcohol and collagen fibers, metal spirals of platinum, tungsten or stainless steel, suture material pieces and detachable balloons, all of which leading to a mechanical obliteration of the vessel volume associated with flow deceleration and subsequent thrombozation.

Both liquid embolizates and particulate embolizates have their specific fields of application.

Generally speaking, liquid embolizates such as fibrin glue, acrylates and Ethibloc® permit a vascular area to be homogeneously filled. This means that a secondary reopening of the embolized area can hardly take place. Moreover, a vascular short circuit in an area previously embolized by means of a liquid embolizate can hardly reopen by a secondary dilatation of neighboring collateral vessels. This is the reason why liquid embolizates are excellently suited for the sclerotization of complex reticular short circuits. Compared to the use of particulate embolizates they offer the advantage that the recanalization risk and frequency is significantly reduced due to the fact that the malformation is filled more completely.

From the range of liquid embolizates acrylates more often than not lead to an irregular filling of the vascular area to be treated which may be the cause of recanalization of the embolized vessel. Moreover, the polymerization of acrylates constitutes an exothermic reaction liberating potentially carcinogenic radicals and monomers.

Fibrin glues have a higher viscosity than acrylates and thus allow the vessel volume to be filled more homogeneously during embolization. When in contact with blood fibrin cross-linking occurs bringing about the desired obliteration of the vessel volume. In the course of a few days, however, the embolizate decomposes relatively quickly so that recanalization may take place in the embolized vessel area before newly formed connective tissue brings about the final obliteration of the vessel.

Ethibloc® is an occlusion emulsion which in its commercially available form consists of 210 mg of zein (prolamine extracted from corn not containing tryptophane and lysine), 162 mg of sodium amidotrizoate tetrahydrate, 145 mg of oleum papaveris (poppy-seeds oil), 316 mg of ethanol and 248 mg of aqua bidestillata per 1 ml of emulsion. Given this composition Ethibloc® has a higher viscosity than acrylates and fibrin glues. It is a corn protein glue dissolved in alcohol which precipitates in the presence of blood or aqueous solutions. The precipitate has a chewing-gum like consistency primarily leading to an occlusion of the vessel lumen. As is evident from postoperative, histological preparations the embolizate injected into a vessel shows a homogeneous filling of the vessel lumen.

Ethibloc® comprises a primary contrast medium (sodium amidotrizoate tetrahydrate) which provides radiopaque characteristics. The content of poppy-seeds oil serves to improve the separation behavior during application. Initially, applications in the field of neuroradiology involved the treatment of highly vascular malignant neoplasms found in the head/neck area, meningiomas and dura angiomas. However, improvements subsequently achieved in the micro-catheter technology as well as embolizate preparation for application via micro-catheters enabled Ethibloc® to be also applied in the area of the brain and spinal cord vessels proper.

The development of micro-catheters of increasingly smaller lumen as well as the application of Ethibloc® in the intracranial area necessitated both modifications of the application technology and improvements of the setting/standardization of Ethibloc®.

It has been described that by adding Lipiodol, an oily contrast medium based on iodinized poppyseed oil, the viscosity of Ethibloc® can be reduced and its radiopaqueness increased. However, such Ethibloc® mixtures have a disadvantage in that they are no longer emulsions but merely suspensions having rapid separation properties. Due to its low specific weight Ethibloc® itself ascends while the specifically heavier Lipiodol collects at the bottom. As far as quick applications are concerned this settling behavior should have less significance but in the event of complicated and time-consuming surgical operations and applications it impairs a homogenous filling of vessels situated in the vascular section to be embolized. The injection of the suspension through the micro-catheter results in small portions of Ethibloc® and Lipiodol alternately exiting via the tip of the catheter and forming blocks or phases consisting of either one or the other material. As a consequence recanalization may occur in the vascular sections to be embolized.

There is another reason why the injection of the suspension via a micro-catheter is to be viewed as problematic. Due to air inclusions developing during mixing and application via micro-catheter air bubbles are automatically formed. Under the influence of the injection pressure exerted such air bubbles are compressed and cause a kind of “air pistol effect” when Ethibloc and Lipiodol mixture is applied which in the most favorable case is just undesirable but may also cause rupturing when the treatment involves thin-walled aneurysms.

On the other hand, however, the higher radiopaqueness of the prolamine, respectively zein emulsion achieved through the addition of Lipiodol is necessary and desired, especially for intracranial applications. Furthermore, the Lipiodol admixture is conducive to the separation of the constituents of the suspension. Bearing this in mind, a higher viscosity which basically also counteracts separation would be desirable as well, in which case, however, due consideration would have to be given to the needs of special applications, in particular if vessels of small diameter are to be embolized, for instance in the area of capillaries and particularly arteriovenous short circuits with reticular (plexiform) short circuiting links existing between the arterial and venous vascular system which require that the viscosity of the embolization medium is lower.

The adjustment of Ethibloc® emulsions to the desired viscosity and radiopaqueness desirable in each individual case has hitherto exclusively been effected with the aid of the commercially available contrast medium Lipiodol.

In the light of these considerations it is the objective of the invention to provide an embolization medium that has the required radiopaqueness and whose viscosity can be set so as to meet the needs of the relevant application, shows no or just a low tendency towards component separation and, all in all, leads to a rapid and uniform precipitation at the application site and enables the vessel lumen in the treatable vascular area to be completely filled to the extent possible.

This objective is reached with a three-component embolizate comprising the constituents named in claim 1.

Preferably, the embolizate according to the invention contains components (b), (c) at a volume ratio ranging between 1:2 and 2:1, with identical volume fractions being especially preferred. It has been found that when the volume relationship of these components is roughly identical, emulsion stability is especially great so that the occlusions produced are particularly uniform.

Moreover, it has turned out to be beneficial to use for the occlusion mixture 30% v/v of component (a) and 15 to 35% v/v each of components (b) and (c). For this purpose component (a) especially consists of a (commercially available and widely applied) zein emulsion in aqueous alcohol. The commercially available zein emulsion additionally contains a conventional radiopaque contrast medium, for example in the form of sodium amidotrizoate tetrahydrate, as well as a vegetable oil to improve the separation characteristics, such as for example poppyseed oil. Such a product is known under the name of Ethibloc®. Instead of the vegetable oil, however, component (a) may contain suitable synthetic oils and, in lieu of the radiopaque contrast medium in the form of sodium amidotrizoate tetrahydrate, suitable other types of known radiopaque contrast agents.

As regards component (b) the liquid radiopaque contrast medium is preferably an agent introduced in medical science under the name of Lipiodol® which is a iodine-containing vegetable oil, i.e. poppyseed oil. Alternatively, other customary radiopaque contrast media may be employed in the usual form, for example an agent which is available under the name of Pantopaque. Preferred are iodine-containing oils but other suitable liquid contrast media may be employed as well which includes agents containing dust or powder of radiopaque materials such as tantalum, platinum or tungsten or other metallic, ionic or non-ionic material in suspended form.

The embolizate according to the invention is produced in a form ready for application especially by homogenization of component (a), mixing of component (b) and admixing of component (c) to prepare a mixture of (a) and (b), in this order, with the requirement that all these steps have to be carried out in the absence of air. Absence of air means that in the form ready for application neither air nor other gases are present in the liquid that may impair the flow characteristics. This may be brought about especially by creating a vacuum but also by evacuating air constituents through centrifuging.

It is of course also feasible to employ other mixing methods providing for the components to be mixed and homogenized in a different order or sequence with the air removal/evacuation being performed prior to, during and subsequent to the mixing process, especially by making use of mechanical systems and equipment.

The components (a), (b) and (c) which are subjected to the mixing process according to the invention to produce the embolizate according to the invention are preferably available separately filled into containers and are intermixed by means of a mixing system. Such a separate filling into containers may, for instance, be arrived at by sterilely drawing up the components into individual syringes as customarily employed in medicine and having them available for introduction via said syringes into the mixing system. Sterilely packed individual packs of the three components may also be employed in separate form or appropriately linked with each other such that they may be opened one into the other and thus enable the mixing process to take place within the packing system. Especially suited is a mixing system consisting of a three-way cock to which the syringes are attached and via which the individual components can be homogenized and admixed by transferring them via the syringes to and fro one after the other. By configuring and tilting the three-way cock in a suitable manner turbulences may be produced that improve the mixing process and homogenization while the components are transferred to and fro through the syringes. Such a three-way cock may be provided with a customary connection system for syringes.

In this respect it is considered beneficial and also the object of the present invention to make available components (a), (b) and (c), in separately packed form at a predetermined volume ratio, together with a mixing system. In particular, such a medical kit also contains an empty pack which is intended to accommodate the prepared mixture. The components and the empty pack consist, preferably, of disposable, single-use syringes. Naturally, the medical kit may also contain an appropriate connection system.

The elimination/exclusion of air can be brought about by creating a vacuum or, alternatively, by placing the syringe or the container accommodating the prepared mixture in a centrifuge.

The embolizate according to the invention is especially used for the occlusion of vessels and vascular malformations. This involves, in particular, aneurysms or arteriovenous short circuits.

Wherever aqueous alcohol or alcohol is referred to in the description this shall be understood to apply to 70 to 96% medical alcohol, as a rule of 96% concentration, and aqua bidestillata. In case the alcohol used is of high concentration the contrast medium of component (b) may contain water or exhibit an increased water content.

Especially preferred at the present time are the following mixtures which are of ascending viscosity in the order they are referred to. As regards stability, these homogeneous Ethibloc-Lipiodol-alcohol emulsions may be improved in that the proportion of Lipiodol is reduced, resp. the proportion of alcohol in the admixture to original Ethibloc is increased in such a manner that the total alcohol content reaches a proportion ranging between 70 and 80% v/v.

1. 1 part of Ethibloc+1 part of Lipiodol+1 part of alcohol

2. 2 parts of Ethibloc+1 part of Lipiodol+1 part of alcohol

3. 4 parts of Ethibloc+1 part of Lipiodol+1 part of alcohol.

If the proportion of Lipiodol in component (b) is low the problem thus caused through the radiopaqueness being impaired may be circumvented in that the oil contained in component (a) serving to improve the separation characteristics is replaced by an increased proportion of a radiopaque contrast medium (for example, sodium amidotrizoate tetrahydrate). A good radiopacity is achieved if the radiopaqueness corresponds to approximately 200 to 350 mg of iodine/ml. Through the admixture of component (c) a useful stabilization of the emulsion can be achieved at any rate. Moreover, the alcohol of course increases the vascular occluding effect due to the vessel wall suffering local damage.

As far as component (a) is a zein emulsion in aqueous ethanol which may possibly contain more customary additives but no radiopaque contrast medium, it may prove expedient to use relatively large volumes, especially of component (b), to enable the required radiopaqueness of the embolizate to be achieved.

If correctly and appropriately prepared the occlusion mixture can be slowly pushed out of the catheter in the form of a (thin) thread which adheres to the wall of the vessel and agglomerates. A separation as encountered with original Ethibloc® could not be observed.

The invention is explained in more detail by way of the following example.

### EXAMPLE

Materials and Method

A total of 236 patients were treated with Ethibloc® embolizates in 458 intervention sessions. Altogether, 1221 arteries of highly vascular pathological vessel lesions were superselectively probed and occluded using micro-catheters.

A total of 173 patients with vascular malformations, 62 with tumors and one patient with an inflammatory illness in the craniospinal area were embolized. Prior to each intervention comprehensive section diagnosis of the region to be embolized was carried out.

Embolization Process

Embolizations were made either transarterially using micro-catheters or in the case of cavernous vascular malformations by direct puncturing during which the embolizate was injected into the target area by means of a puncturing needle. The transarterial embolizations by micro-catheter were effected via an access to the arteria femoralis. Diagnostic angiography of the target site was again performed before the micro-catheter was introduced coaxially into the main artery leading to the vascular area to be embolized. The correct position of the micro-catheter tip was checked via the micro-catheter by performing superselective angiography in series.

As regards the individual embolizate mixtures used efforts were made to introduce the embolizate into the angiom nest and tumor nest in the best possible way. Having positioned the micro-catheter optimally one of the following embolizate emulsions were prepared:

1. Genuine Ethibloc® emulsion (undiluted Ethibloc®)

2. Ethibloc® suspension prepared through mixing Ethibloc® with Lipiodol at a ratio of 1:1 (old mixing ratio)

3. Ethibloc® emulsion prepared by mixing Ethibloc®, Lipiodol and alcohol at a ratio of 1:1:1 (mixture according to the invention).

Having connected the Ethibloc® syringe with another 10-ml luerlock syringe via a three-way nylon cock at an angle of 90° the embolizate, after the entire volume of air has been evacuated, is homogenized by alternately transferring it to and fro through the syringes until the mixture shows a uniform yellowish color. Following this, a 1-ml luerlock glass syringe is connected to the three-way cock. With air being evacuated simultaneously the intended embolizate volume is drawn up into the 1-ml luerlock syringe. For the production of the “old mixture” exclusively Lipiodol is mixed with Ethibloc® using a 1-ml or 0.5-ml luerlock syringe and the three-way cock with said mixture then being directly used for the embolization process.

For the production of the embolizate according to the invention (new mixture) the relevant proportion of Lipiodol is supplemented by pure alcohol (96% medical alcohol) of equal proportion. In this way a stable emulsion is obtained. The color of the embolizate during mixing changes from vivid yellow to a pale or almost whitish yellow. The embolizates according to the invention have shown stability over a sufficiently long period, i.e. no phase separation was observed over a period of 2 to 2 hours.

The ultimately prepared embolizate was subsequently applied in a traditional manner via micro-catheter under angiographic supervision, with efforts being made to quickly fill the dead space in the micro-catheter and slowly inject the embolizate into the probed vessel. In each case it could be observed that the embolizate according to the invention offered better flow characteristics, and no phase separation occurred as was encountered with the “old mixture” during application. In each case, the vessel could be filled homogeneously, quickly and uniformly with the embolizate according to the invention. When using the old mixture a tendency towards phase separation could be observed in each case accompanied by the formation of individual Ethibloc droplets and Lipiodol droplets.

Results

In comparison to alternative liquid embolizates Ethibloc® is an embolizate that can be employed for the treatment of a variety of vascular morphologies and flow patterns. However, the application of pure Ethibloc® in the manner and of the nature furnished by the manufacturer will not be sufficient for this purpose. For the embolization of minute vessels Ethibloc® is beneficially to be diluted with Lipiodol and, in particular, with Lipiodol plus additional alcohol.

In the framework of the invention the mixture according to the invention leads to the viscosity of the embolizate to be reduced and allows the embolizate to ingress more deeply into the arteriovenous short circuit until venous embolization is reached. In contrast thereto a proximal vessel occlusion occurs primarily as a rule during the embolization of reticular arteriovenous short circuits.

A preceding embolization by means of occlusion spirals made of platinum may prove advantageous and result in the embolizate to be better anchored at the application site. Platinum spirals applied to the treatment of arteriovenous malformations produce a lattice structure which is then bonded with the aid of Ethibloc®.

1. Liquid embolizate, in particular for the occlusion of vascular malformations, consisting of
(a) 20-80% v/v of an occlusion mixture containing a zein emulsion in aqueous ethanol,
(b) 10-40% v/v of a radiopaque contrast medium in liquid form and
(c) 10-40% v/v of ethanol,
with components (a), (b) and (c) being available separately and mixed, immediately before application takes place, to form a homogenous emulsion.

2. Embolizate according to claim 1, characterized in that components (b) and (c) are provided at a volume ratio ranging between 1:2 and 2:1, preferably at equal volume fractions.

3. Embolizate according to claims 1 or 2, characterized in that it consists of 30-70% v/v of component (a) and of 15-35% v/v each of components (b) and (c).

4. Embolizate according to any of the above claims, characterized in that component (a) contains a zein emulsion in aqueous alcohol, a radiopaque contrast medium, and a vegetable oil.

5. Embolizate according to any of the above claims, characterized in that component (a) consists of Ethibloc® and component (b) of Lipiodol®.

6. Embolizate according to any of the above claims, in a form ready for application, produced by homogenization of component (a), admixing with it component (b) and admixing of component (c) with the mixture comprising (a) and (b), with all these steps being carried out in the absence of air.

7. Method for the production of a liquid embolizate according to any of the above claims, characterized in that, in the absence of air, component (a) is homogenized, component (b) admixed with component (a) and component (c) is admixed to the mixture comprising components (a) and (b).

8. Method according to claim 7, characterized in that the mixing process takes place under vacuum.

9. Method according to claim 7, characterized in that the elimination of air is achieved by centrifuging.

10. Method according to claim 7, characterized in that components (a), (b) and (c) are available in separately packed form and are intermixed with the help of a mixing system.

11. Method according to claim 10, characterized in that the components (a), (b) and (c) are made available drawn-up into syringes.

12. Method according to claim 10 or 11, characterized in that the mixing system is a three-way cock.

13. Application of the embolizate according to any one of the claims 1 to 6 for the occlusion of vessels and vascular malformations.

14. Application according to claim 9, characterized in that the vascular malformations are aneurysms or arteriovenous short circuits.

15. Medical kit for application according to claims 9 or 10, characterized in that said kit contains the components (a), (b) and (c) according to any one of the claims 1 to 5 at a predetermined volume ratio as well as a mixing system.

16. Medical kit according to claim 15, characterized in that said kit contains the components (a), (b) and (c) drawn up into disposable, single-use syringes, a three-way cock and at least one empty syringe which accommodates the readily prepared mixture.
